(19) United States
(12) Patent Application Publication
(10) Pub. No.: US 2007/0083163 A1
(43) Pub. Date: Apr. 12, 2007

(54) IV CUFF

(76) Inventor: Candace Rydell, Tampa, FL (US)

Correspondence Address: CHRISTOPHER PARADIES, PH.D., FOWLER WHITE BOGGS BANKER, P.A., 501 E KENNEDY BLVD, STE. 1900, TAMPA, FL, 33602, US

(21) Appl. No.: 11/467,321

(22) Filed: Aug. 25, 2006

Related U.S. Application Data
(60) Provisional application No. 60/711,854, filed on Aug. 26, 2005.

(51) Int. Cl. A61M 5/32

(52) U.S. Cl. 604/174; 604/179

(57) ABSTRACT

A cuff for use with an intravenous line, tubing and associated dressing for patients undergoing treatment conceals an intravenous line and associated dressing when fitted on a limb and includes a pocket. The pocket may be formed in the cover or is attached to the cover. In one example, such a pocket may house or support containers such as medication containers which may deliver drug therapy such as antibiotics. In one example, the medication containers may be then attached to the IV lines.

### FIELD OF THE INVENTION

The field relates to covers for use in catheters and other types of tubing, including tubing lines, used in intravenous therapy and includes other therapies in which fluids are needed for any period of time, including extended period of time.

### BACKGROUND

Intravenous therapy involves administration of liquid substances into the vein of a patient. There are many forms of IV (intravenous) therapy. For long term usage in patients, a peripherally inserted central catheter line (PICC lines) may be one modality of IV therapy. A PICC line is inserted into a peripheral vein, often in the arm, and then is drawn upward until the catheter is in the superior vena cava of the heart.

However, the IV lines may need support and its attachments which may include medication bags often dangle from such lines. Matyas, U.S. Pat. No. 5,885,254 and Warner, U.S. Pat. No. 6,276,364 describe protective covers or sleeves for arms containing an IV site which includes PICC lines. They are often made of waterproof, polymeric materials but may not be aesthetically pleasing for patients such as young children.

There have been other ways for protective covers for use in patients, including young children, who undergo long term therapy, which may involve PICC lines. Harris, U.S. Pat. No. 6,540,724, describes a self-supporting fabric catheter cover for covering the external tubes of an intravenous therapy device. Such a cover would include releasable securing means such as hook and loop fasteners. Villiapiano, U.S. Pat. No. 6,032,289, as well, describes fabric covers that include sleeves worn over the arm but again includes fastening means such as hook and loop fasteners. However, such covers may be problematic in that they are typically fastened. If a cover causes restricted movement, such a cover may be akin to a brace in which movement is limited.

There needs to be protective covers that are not only aesthetically pleasing but also be more easily worn.

### SUMMARY OF THE INVENTION

A cuff is used for concealing PICC lines, tubing and/or sterile dressings in intravenous therapy, chemotherapy, or where intravenous connections are needed by a person for an extended period of time. In one example, the cuff is slipped over the arm of the user and has a tubular form closed without the need of any fastener. The tubular cuff may be formed by joining two edges of a sheet of material into a tubular form or by forming a seamless tube. In either case, the tubular cuff is of a stretchable material capable of sliding onto a limb of the patient and remaining in a fixed position on the limb by tensile forces of the resilient material. Preferably, the material applies only a gentle tension that does not restrict the patient's limb or circulation, allowing the cuff to be in place during therapy. The cuff may then be slid over the arm in order to cover the tubing and related attachments of a PICC line.

The cuff includes a pocket for protecting items connected to IV tubing lines. In one example, the IV tubing lines include PICC lines and a related PICC attachment inserted in the patient's limbs such as an arm or leg. The attachment is inserted through or into the pocket which may be formed in the cuff or may be attached to the cuff.

One object of the cuff is to provide better aesthetics and comfort to patients, including children. Still another object of the cuff is to allow for the IV tubing and related attachments to be protected during and between treatments without restricting freedom of movement of the patient.

### BRIEF DESCRIPTION OF THE FIGURES

The drawings describe some examples of the features of a cuff, as shown.

FIG. 1 illustrates one example of a cuff.

FIG. 2 depicts another example of a cuff.

FIG. 3 shows another example of a cuff.

FIGS. 4A, 4B, and 4C illustrate another example of a cuff in A) a perspective view; B) an end view; and C) a perspective view of the cuff turned inside out.

FIGS. 5A, 5B and 5C illustrate perspective, front, and back views of a cuff having a tubular shape formed by attaching two sides of a cover.

### DETAILED DESCRIPTION OF PREFERRED EMBODIMENTS OF THE INVENTION

The examples described and the drawings rendered are illustrative and are not to be read as limiting the scope of the invention as it is defined by the appended claims.

In an example illustrated in FIG. 1, a cuff for use on a patient's limb, such as an arm or leg has a cover 12 made of a resilient, flexible material formed into a tube by joining two edges (not depicted) at a seam 14. The circumference of the tube and the tensile properties of the stretchable material, such as a spandex material such as Lycra,® or other stretchable fabric, provide a snug fit on the arm or leg of a patient. Such a material helps retain the cuff in a fixed position after sliding the cuff onto the limb.¹ The cuff may be made of materials suitable for matching with other articles of clothing. Such materials may include materials having the outward appearance of cotton, nylon, polyester or acrylic but are not limited to such materials. Additionally, the entire cuff may be made of elastic and flexible materials but is not limited to such materials. Materials are selected such that they do not restrict the wearer's freedom of movement. The cover may include a water resistant, breathable layer. Waterproof materials cannot be used, because the cuff is for wearing throughout the day and waterproof materials are not always suitable for such use because they cause conditions favorable for infection or skin disorders.
¹Lycra® is a registered trademark of Invista North America S.A.R.L.

The cuff may include antimicrobial properties. In one example, the antimicrobial properties are provided by Microban,® an antimicrobial additive used in textiles and the like.² Antimicrobial materials may be selected such that a cuff is reuseable and prevents infectious microorganisms from causing infection.
²Microban® is a registered trademark of Microban Products Company.

Preferably, the cuff includes a cover comprised of a flexible material. As shown in FIG. 1, one end of the tubular cover 12 is wider than the other end, forming a tapered, tubular structure, for example. The tapered tubular structure of the cover 12 is suitable for use on a forearm or leg. In one embodiment, the shape of the tubular cover 12 may be contoured to fit the anatomy of an arm or leg. In another example, the tubular cover is not tapered or contoured.

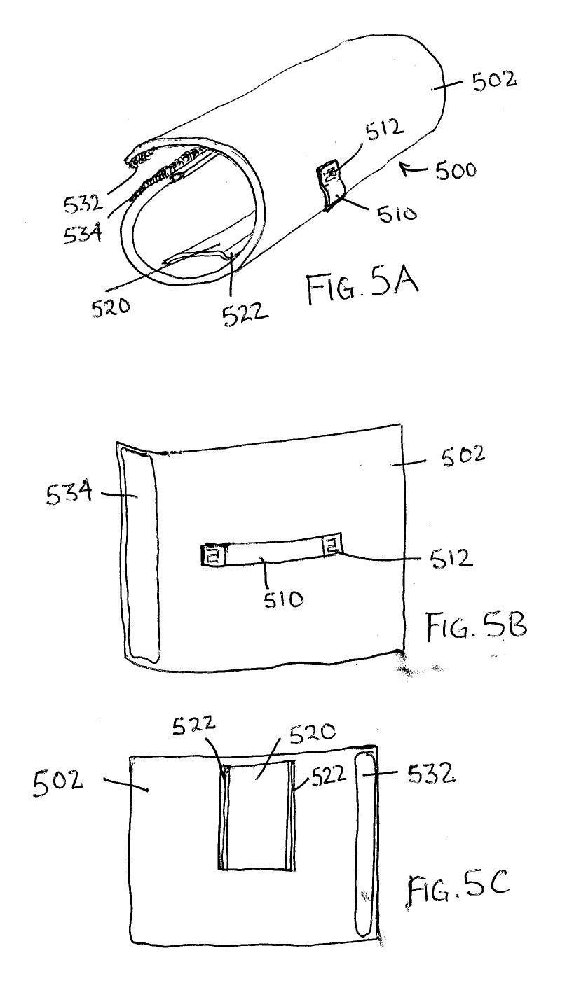

The cover 12 may be sized to fit patients of different ages and sizes. The overall circumference of the tubular cover in FIG. 1 is sized less than the circumference of the arm such that tensile forces hold the cover 12 in place without restricting movement or circulation substantially. In one example, a cover 12 is made of exceptionally resilient and flexible materials, spandex materials such as Lycra® such that one size of a cuff fits all patients.³ In another example, such as shown in FIGS. 5A-5C, a fastener is used to make the cuff adjustable. In the example of FIGS. 5A-5C, the cover 502 is still made of a flexible material that provides an unrestricted freedom of movement and range of motion to the wearer. In one example, the cover 502 is a loosely knit material that substantially conceals PICC lines while providing freedom of movement.
³Lycra® is a registered trademark of Invista North America S.A.R.L.

An IV site may contain lines, and a mechanism for connecting to lines and attachments. The cover 12 of FIG. 1 has a surface area sufficient to conceal tubes, dressings, and attachment mechanisms. In one example, a PICC line with an attachment mechanism is concealed by the cuff. Attachments may include an attachment for injection of medications. In one example, the attachment is a medication container.

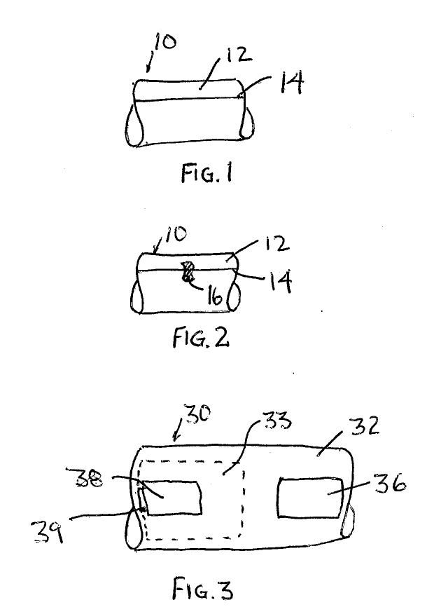

In the example of FIG. 2, the depicted cuff 10 has an additional feature not found in FIG. 1. A pocket 16 is formed by a patch attached to the outer surface of the cover 12. In this example, the pocket 16 extends over a portion of the seam 14, but the pocket 16 may be disposed anywhere on the surface of the cover 12. In the example of FIG. 2, a slot or opening is left in the seam 14 under the pocket 16. The slot or opening (not shown but readily understood) is concealed by the pocket 16. The pocket 16 may have one or both ends open and may be fixedly attached or attached by a fastener, such as hook and loop fasteners, such that the material of the pocket 16 may be disposable and or be sterile. Using the slot or opening, an intravenous line leading to the patient may be inserted through the slot or opening and secured within the pocket 16.

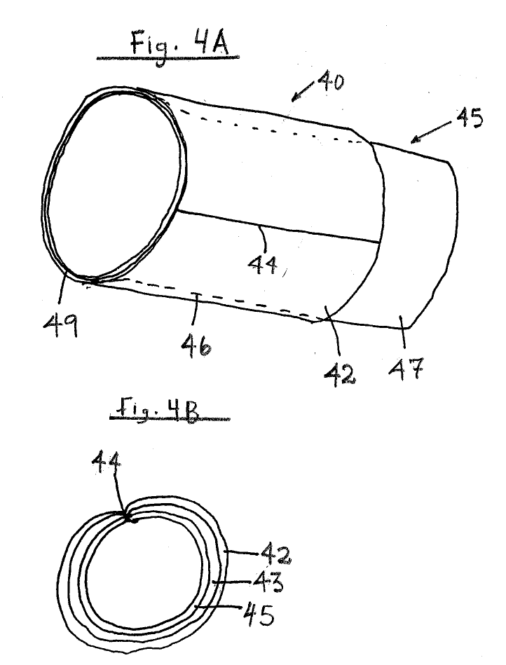

In the example of FIG. 3, additional features which are not shown in FIGS. 1 and 2 are illustrated. The cuff 30 is seamless, and is being formed as a tubular material without a seam. A pocket 36 is attached to the outer surface of the cover 32. A second pocket 38 is attached at an opposite end of the cover 32. The pockets 36, 38 may be open at one or both ends and may used to secure lines and attachments for intravenous treatments. The second pocket 38 is attached over a third pocket 39. These pockets 36, 38, 39 may be of any size and shape. Pass-through holes may be made through the cover 32 and between pockets 38, 39 as needed for use in treatments.

Additionally, the cuff 30 has an inner pocket 33 (hidden lines shown dashed), formed on the inner surface of the cuff 30. The inner pocket 33 may have one end open or two opposite ends open, as previously described for exterior pockets 36, 38, 39. If both ends are open, then no pass through hole or slot is needed, and a PICC line may be inserted in one end and out the other. The pockets may be fixedly attached or removably attached. If removably attached, the pockets may be of a disposable, sterile material, which may be of a color and texture similar to the cover 32.

In other examples, the cuff may only have one inner pocket, which may have one end open or two opposite ends open.

A pocket may house and further support attachments to the IV line. In one example, the PICC line may include a PICC apparatus for connecting to a medication container. Such attachments may include, without limitation, medications such as antibiotics.

A cuff may be worn over the upper extremity of the arm, above the elbow region or elsewhere on an arm or leg, so long as it is properly sized. A cuff may even be worn on a torso, such as a tube top, as an item of clothing, if properly sized. The cover may be made of materials suitable for clothing and may be worn like any clothing.

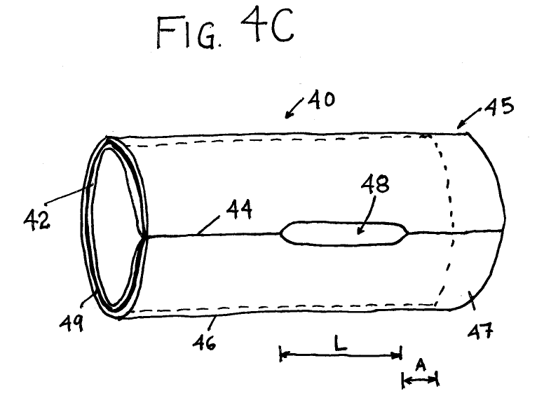

In the examples of FIG. 4A-4B, additional features of a cuff 40 are illustrated. A portion 47 of a sleeve 45 extends from the cover 42. In this example, the cuff 40 has a seam 44 or the cover 42 may be seamless. For example, a second portion 46 of the inner sleeve 45 is concealed within the cover 42, which is represented by broken lines. The second portion 46 may be attached to the cover 42 along the longitudinal seam 44. In one example, a slot 48 such as illustrated in FIG. 4C is formed in the sleeve 45 along one edge of the sleeve 45, such that an IV tube extending from a patient's arm may be inserted through the slot 48, extending into the pocket 43. A distance A from an end of the slot to the end of the cover may be selected to allow the IV tube to extend beyond the cover 42, when the cover 42 is pulled up the arm. The slot length L may be selected such that the cover 42 conceals a PICC line, when the cover 42 is pulled forward on the arm while keeping the PICC line from being covered by the inner sleeve 45.

The inner sleeve 45 may also be attached to the cover 42, elsewhere, and may also have a slot formed elsewhere in a portion of the sleeve in alternative embodiments not illustrated in FIGS. 4A-C. The inner sleeve 45 is joined to the cover 42 at the end of the second portion 46, which may be coterminous with the end of the cover 42. Thus, the sleeve 45 and the cover 42 form a pocket 43, as shown in FIG. 4B. If the end of the second portion 46 of the inner sleeve 45 is joined coterminously with the cover 42, then the pocket 43 has a closed end 49 and an open end corresponding to the end of the cover 42. The pocket 43 may also have two open ends, if the inner sleeve 45 is not joined coterminously with the cover 42. However, a pocket 43 with a closed end has advantages for applications that use the pocket 43 as a compartment for storing and securing tubing and the like.

In another example, an opening is provided in a portion of the end 49 of the inner sleeve 45 joined coterminously with the cover 42. In yet another example, the end of the inner sleeve 45 may be joined at a location other than the end of the cover 42. Various other embodiments may include alternative configurations as shown and suggested in the drawings and examples provided.

In FIG. 5A, a pocket is shown extending about halfway along the inner surface of a cover, and the inner sleeve 45 of FIGS. 4A and B may also be so joined. Furthermore, additional pockets (not shown) may be defined by additional longitudinal seams (not shown) joining the inner sleeve 45 to the cover 42.

FIGS. 5A-5C illustrate an example of a resilient, flexible cuff 500 having an adjustable cover 502, which is formed into a tube using an attachment mechanism 532, 534. In this example, the attachment mechanism is a hook and loop attachment mechanism 532, 534, which provides for some adjustment of the circumference of the tubular cover 502. An inner pocket 520 has two open ends and extends about halfway along the inner surface of the cover 502. The pocket 520 is attached to the cover 502 along two sides 522, which may be fixedly or removably attached. A strap 510 is removable attached, using a magnetic attachment 512 mechanism on the outer surface of the cover 502.

Alternative combinations and variations of the examples provided will become apparent based on this disclosure. It is not possible to provide specific examples for all of the many possible combinations and variations of the embodiments described, but such combinations and variations may be claims that eventually issue.

What is claimed is:

1. A device for use with an intravenous line, tubing, and associated dressing for patients undergoing treatments comprising:
a tubular cover being made of a material capable of concealing the presence of the intravenous line and associated dressing when fitted on a limb; and a pocket wherein the pocket is formed in the cover or attached to the cover.

2. The device of claim 1, wherein the cover forms a closed tube without a fastener and is tapered from a first end to a second end.

3. The device of claim 1, wherein the material of the cover is stretchable, such that the cover is capable of being slipped onto a limb and remains fitted in place on the limb by the elasticity of the stretchable material.

4. The device of claim 1, wherein the cover is shaped to conform to the anatomy of a patient's limb, such that tension of the material does not substantially affect circulation in the limb and the cover is capable of being left in place during treatments.

5. The device of claim 1, wherein the pocket is attached to the cover.

6. The device of claim 5, wherein the pocket is removably attached on an outer surface.

7. The device of claim 6, wherein the pocket is of a disposable, sterile material capable of being used to protect the intravenous line.

8. The device of claim 6, wherein the pocket is of a material and design matching the material of the cover.

9. The device of claim 1, wherein the pocket is formed in the cover.

10. The device of claim 9, wherein the pocket is concealed by the cover.

11. The device of claim 1, wherein the tubular cover is of a stretchable material and is fitted on the patient by placing the device around a portion of the patient's anatomy and closing a fastener, binding one edge of the cover to another edge of the cover.

12. The device of claim 11, wherein the fastener is a hook and loop fastener and the stretchable material provides unrestricted movement of the patient.

13. The device of claim 1, further comprising a second pocket.

14. The device of claim 1, wherein the cover is seamless.

15. The device of claim 1, further comprising a strap attached to the cover.

16. The device of claim 15, wherein the strap is disposed in a pocket.

17. The device of claim 9, wherein the pocket is formed by an inner sleeve attached to an inner surface of the cover.

18. The device of claim 17, wherein a portion of the inner sleeve extends from the cover.

19. The device of claim 17, wherein the inner sleeve is attached to the inner surface of the cover along a longitudinal seam;
the longitudinal seam attaching two edges of the cover, forming a tubular shape; and a slot is defined by an opening in a portion of an edge of the inner sleeve.

20. The device of claim 19, wherein a portion of the inner sleeve extends from the cover, and an end of the inner sleeve opposite of the portion is attached to the cover, closing the pocket.

21. The device of claim 20, wherein a position and length of the slot is selected such that both the length of the slot and a distance from one end of the slot and an open end of the pocket are less than a length of a standard peripherally inserted catheter line used for therapy for the patients undergoing treatments.

22. The device of claim 21, wherein the cover has antimicrobial properties.
